(19) United States
(12) Patent Application Publication
(10) Pub. No.: US 2021/0275154 A1
(43) Pub. Date: Sep. 9, 2021

(54) BONE BIOPSY DEVICE AND RELATED METHODS

(71) Applicant: Merit Medical Systems, Inc., South Jordan, UT (US)

(72) Inventors: Robert Bilgor Peliks, San Francisco, CA (US); Jeremy Snow, South Jordan, UT (US); Jade Ollerenshaw, North Melbourne (AU)

(21) Appl. No.: 17/190,123

(22) Filed: Mar. 2, 2021

Related U.S. Application Data
(60) Provisional application No. 62/984,685, filed on Mar. 3, 2020.

(51) Int. Cl. A61B 10/02

(52) U.S. Cl. CPC A61B 10/025; A61B 2010/0258; A61B 10/0266

(57) ABSTRACT

Devices and methods used to obtain core tissue samples are disclosed. The devices may be configured to drill into cortical bone and saw a hole into a bone lesion and/or bone marrow while obtaining the core tissue sample. The devices can include a motor and a transmission configured to rotate a trocar having a tip configured for drilling and an outer coax cannula having a cutting tip configured for sawing. The core tissue sample may be received within an inner cannula. A part-off tab may cut or sever the core tissue sample from the bone lesion and/or bone marrow.

### RELATED APPLICATIONS

This application claims priority to U.S. Provisional Application No. 62/984,685, filed on Mar. 3, 2020 and titled, “BIOPSY DEVICE AND RELATED METHODS,” which is hereby incorporated by reference in its entirety.

### TECHNICAL FIELD

The present disclosure relates generally to devices used to perform a biopsy procedure, specifically a bone biopsy procedure. More specifically, the present disclosure relates to devices used to drill into a bone to obtain a core tissue sample of a bone lesion and/or bone marrow.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a perspective view of an embodiment of a bone biopsy device.

FIG. 2 is a perspective exploded view of the bone biopsy device of FIG. 1.

FIG. 3A is a perspective view of the bone biopsy device of FIG. 1 in a trocar extended configuration with a portion of a handle housing removed.

FIG. 3B-1 is a rear perspective view of the bone biopsy device of FIG. 1 in a trocar retracted configuration with a portion of a handle housing removed.

FIG. 3B-2 is a front perspective view of the bone biopsy device of FIG. 1 in a trocar retracted configuration with a portion of a handle housing removed.

FIG. 3C is a front perspective view of the bone biopsy device of FIG. 1 in a part-off tab actuated configuration with a portion of a handle housing removed.

FIG. 4 is a perspective view of an inner cannula of the bone biopsy device of FIG. 1.

FIG. 5 is a perspective view of an intermediate cannula of the bone biopsy device of FIG. 1.

FIG. 6A is a perspective view of an outer coax cannula of the bone biopsy device of FIG. 1.

FIG. 6B is a perspective view of a cutting tip of the outer coax cannula of FIG. 6A.

FIG. 7A is a perspective view of an embodiment of a trocar of the bone biopsy device of FIG. 1.

FIG. 7B is a perspective view of another embodiment of a trocar of the bone biopsy device of FIG. 1.

FIG. 8A is a cutaway side view of a part-off tab of the intermediate cannula of FIG. 1 in a part-off tab unactuated configuration and a trocar extended configuration.

FIG. 8B is a cutaway side view of the part-off tab of the intermediate cannula of FIG. 1 in the part-off tab unactuated configuration and a trocar retracted configuration.

FIG. 8C is a cutaway side view of the part-off tab of the intermediate cannula of FIG. 1 in a part-off tab actuated configuration and the trocar retracted configuration.

FIG. 9A is a side view of the bone biopsy device of FIG. 1 ready for use.

FIG. 9B is a side view of the bone biopsy device of FIG. 1 inserted into a patient's skin over a guidewire.

FIG. 9C is a side view of the bone biopsy device of FIG. 1 drilled through a cortical bone layer.

FIG. 9D is a side view of the bone biopsy device of FIG. 1 drilled into a bone lesion and/or bone marrow to obtain a core tissue sample.

FIG. 9E is a side view of the bone biopsy device of FIG. 1 with the inner cannula, intermediate cannula, and trocar removed from an outer coax cannula.

FIG. 9F is a side view of the bone biopsy device of FIG. 1 with an outer coax cannula removed and a tissue sample being ejected from the inner cannula.

FIG. 9G is a side view of the bone biopsy device of FIG. 1 with the inner cannula, intermediate cannula, and trocar removed from an outer coax cannula and an aspiration needle inserted through the outer coax cannula.

FIG. 9H is a perspective view of a trocar assembly.

### DETAILED DESCRIPTION

A bone biopsy device may include a handle assembly, a coax assembly, and a power pack. The handle assembly may include a housing configured to hold an inner cannula. The inner cannula may extend distally from the housing and may be configured to receive a core tissue sample. A trocar with a penetrating tip may be slidably disposed within a lumen of the inner cannula. The housing may include an extension member that is configured to displace the trocar relative to the inner cannula from a retracted configuration to an extended configuration where the trocar can drill into a bone. A motor and a transmission may rotate the trocar. In certain instances, the transmission may include a worm drive. In other instances, the transmission may include a plurality of spur gears. The inner cannula and trocar may be configured to remain part of the handle assembly (e.g., coupled to the housing) before, during, and after a biopsy procedure. The coax assembly may be selectively detachable from the handle assembly. The coax assembly may include an outer coax cannula extending distally from a coax connector. The inner cannula may be partially disposed within a lumen of the outer coax cannula. The outer coax cannula can be rotated by the motor. A tip of the outer coax cannula may be a cutting tip (e.g., a trephine tip) and be configured to saw into a bone lesion and/or bone marrow. The power pack may be selectively removable from the handle assembly such that the power pack may be a reusable component. The power pack may comprise a power source, a controller, and a connector. The power pack and/or controller may also comprise a printed circuit board. In some instances, the motor may also be selectively removable from the handle assembly such that the motor may also be a reusable component (for instance, the motor may be selectively removable with the power pack).

The bone biopsy device may be used by a practitioner to obtain a core tissue sample of a bone lesion and/or bone marrow. In other instances, the bone biopsy device may be used to obtain a core tissue sample of other tissues within a patient, such as a soft tissue sample. In use, the trocar and outer coax cannula may be rotated by the motor and drilled into the cortical bone layer adjacent to a lesion and/or bone marrow. The trocar may be retracted, and the outer coax cannula rotated to saw a core tissue sample of the lesion and/or bone marrow that is collected in the inner cannula. The outer coax cannula may be removed from the inner cannula and an intermediate cannula can be used to actuate a part-off tab to aid in retaining a core tissue sample within the inner cannula. The trocar can later be advanced within the inner cannula to eject the core tissue sample. A needle or aspiration needle can also be inserted into the outer coax cannula to collect or aspirate bone marrow, blood, and/or tissue cells. A needle could also be inserted into the outer coax cannula to infuse or inject a substance (such as a medicament) into the patient.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure but is merely representative of various embodiments. While the various aspects of the embodiments are presented in drawings, the drawings are not necessarily drawn to scale unless specifically indicated.

It will be appreciated that various features are sometimes grouped together in a single embodiment, figure, or description thereof for the purpose of streamlining the disclosure. Many of these features may be used alone and/or in combination with one another.

The phrase “coupled to” refers to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest the practitioner during use. As specifically applied to the bone biopsy device, the proximal end of the device refers to the end nearest the handle and the distal end refers to the opposite end, the end nearest the end of the outer coax cannula. Thus, if at one or more points in a procedure a physician changes the orientation of the device, as used herein, the term “proximal end” always refers to the handle end of the device (even if the distal end is temporarily closer to the physician).

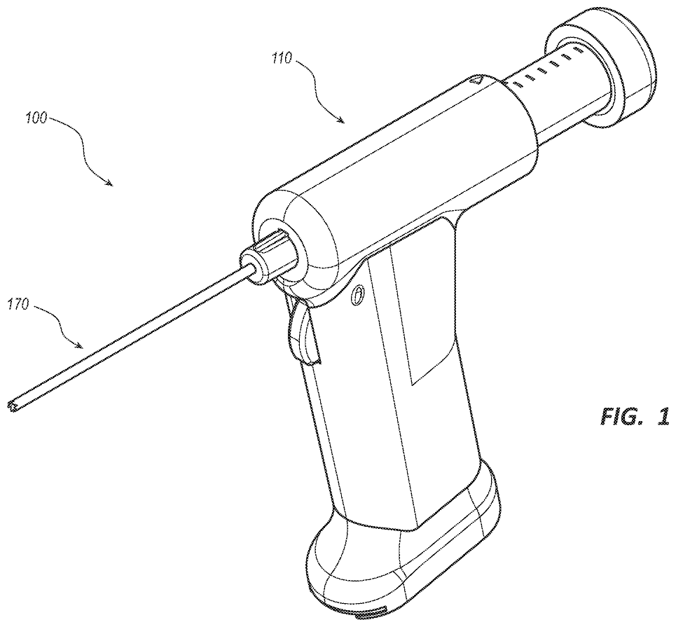

FIGS. 1-9H illustrate different views of bone biopsy devices and related components. In certain views each device may be coupled to, or shown with, additional components not included in every view. Further, in some views only selected components are illustrated, to provide detail into the relationship of the components. Some components may be shown in multiple views, but not discussed in connection with every view. Disclosure provided in connection with any figure is relevant and applicable to disclosure provided in connection with any other figure or embodiment.

FIGS. 1-9G depict one embodiment of a bone biopsy device 100. The bone biopsy device 100 includes a handle assembly 110 and a coax assembly 170 as illustrated in FIG. 1.

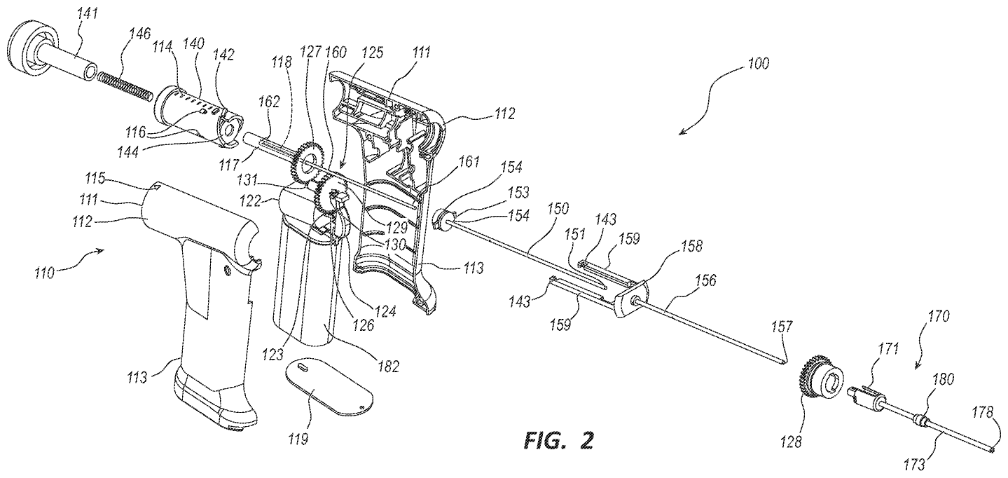

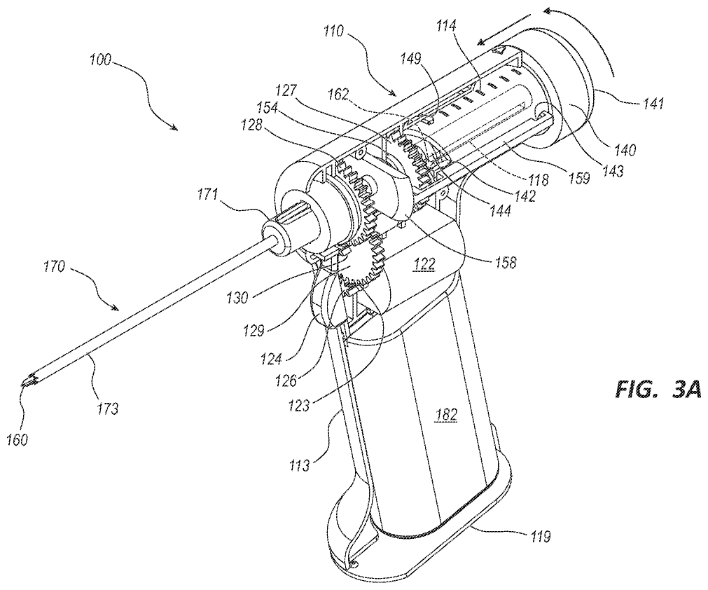

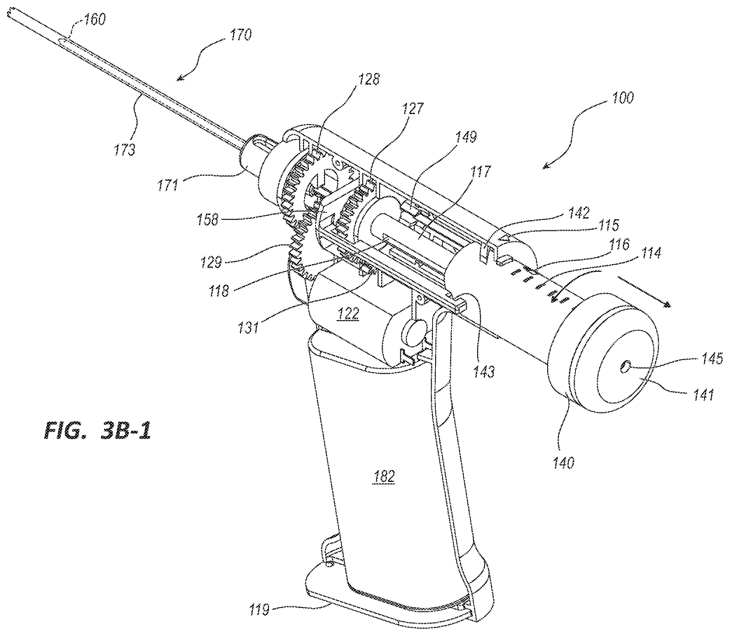

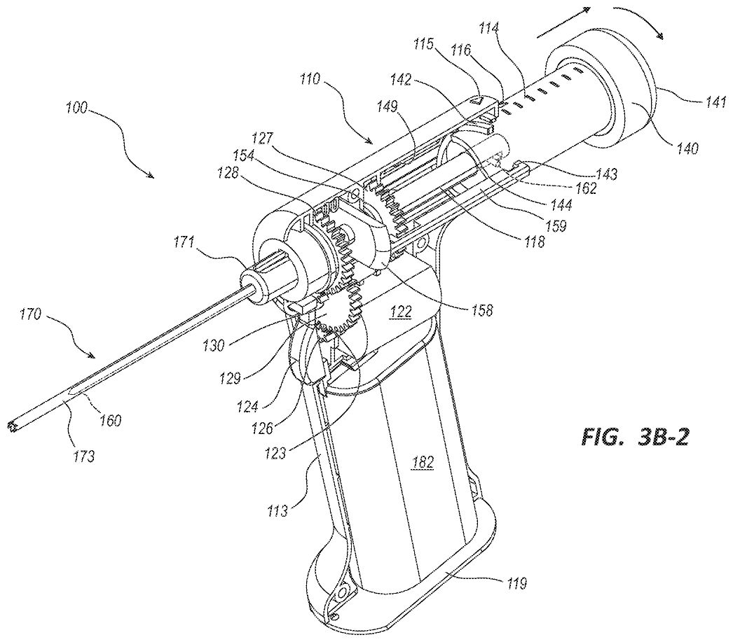

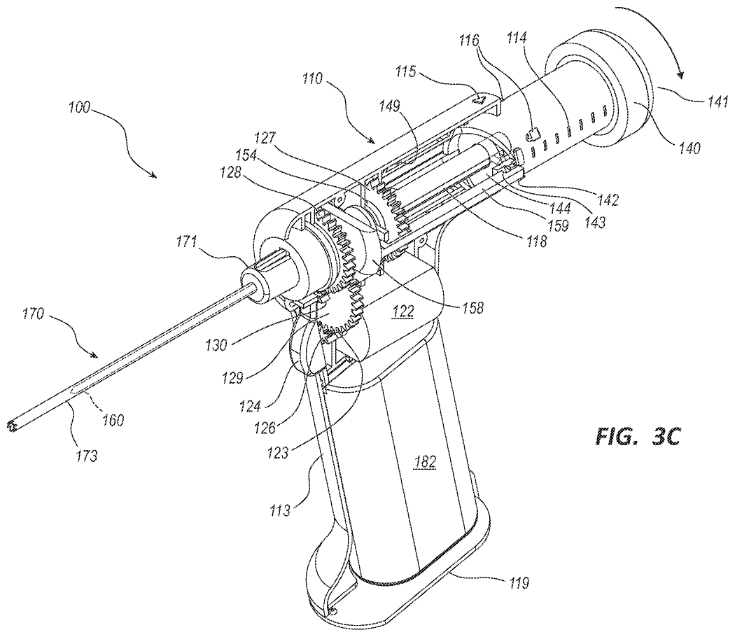

As depicted in an exploded view of the bone biopsy device 100 of FIG. 2 and perspective views of FIGS. 3A-8, the handle assembly 110 may at least partially include a handle housing 111, a motor 122, a motor activation switch 124, a transmission 125, an inner cannula 150, an intermediate cannula 156, a penetrating member or trocar 160, and a power source 182. The handle housing 111 can include an upper portion 112 and a grip portion 113. The grip portion 113 may be configured to be grasped by a hand of a practitioner during use of the bone biopsy device 100. The motor activation switch 124 may be disposed adjacent a distally facing surface of the grip portion 113 such that the motor activation switch 124 may be engageable by a finger of the practitioner. In other embodiments, the motor activation switch 124 may be disposed on any other suitable surface of the handle housing 111. The motor activation switch 124 may be operably coupled to a sensor, such as a linear potentiometer, one or multiple limit switches, photo interrupters, reed switches, a hall sensor, etc. The sensor may be operably coupled to a control member that may control a rotation speed of the motor 122. The rotation speed of the motor 122 may include two discrete speeds, three discrete speeds, four discrete speeds, or more discrete speeds. In another embodiment, the rotation speed of the motor 122 may be variable up to about 50,000 rpm. In other embodiments, the rotation speed of the motor 122 may be controlled using pulse width modulation, voltage drop, or any other suitable technique.

The handle housing 111 may be formed of two separate halves that may be coupled using any suitable technique. For example, the separate halves may be coupled using a snap fit, welding, gluing, fasteners, pins, etc. The handle housing 111 may include any suitable polymeric and/or metallic material, such as polycarbonate, acrylonitrile butadiene styrene, polycarbonate acrylonitrile butadiene styrene copolymer, nylon, acetal, polyethylene (e.g., such as high-density polyethylene and/or low-density polyethylene), silicone, thermoplastic elastomers, steel, stainless steel, aluminum, ceramic, and combinations thereof. The polymers may also be reinforced with other materials, such as glass or aramid fibers. The handle housing 111 may be formed using any suitable technique, such as injection molding, thermoforming, machining, 3D printing, etc. The handle housing 111 can include a plurality of pockets or recesses configured to hold or retain at least some of the components of the handle assembly 110.

In the depicted embodiment, the motor 122 may be disposed within the grip portion 113 of the handle housing 111. The motor 122 may be any suitable type of rotatory motor. For example, the motor 122 may be a DC brushed motor, a DC brushless motor, a stepper motor, a servo motor, a pneumatic motor, or an AC powered motor, etc. The motor 122 may also be bi-directional. The motor 122 can include a drive shaft 123 extending from the motor 122. The motor 122 may rotate the drive shaft 123 at a speed ranging from about 0 rpm to about 50,000 rpm, or from about 15 rpm to about 20,000 rpm. The motor 122 can be electrically coupled to the power source 182 and to the motor activation switch 124.

As illustrated, the transmission 125 can be primarily disposed within the grip portion 113 of the handle housing 111. The transmission 125 can be operably coupled to the motor 122. In the illustrated embodiment, the transmission 125 includes a pinion gear 126, a reduction gear 129, a trocar gear 127, and a coax gear 128. The pinion gear 126 can be fixedly coupled to the drive shaft 123 and rotated by the motor 122. The reduction gear 129 can engage with and be driven by the pinion gear 126. In other embodiments, the transmission 125 may not include the reduction gear 129 such that the pinion gear 126 engages directly with the trocar gear 127 and the coax gear 128.

The reduction gear 129 is shown to include a distal pinion gear 130 and a proximal pinion gear 131 disposed at a proximal end of a shaft. The distal pinion gear 130 can engage with and drive the coax gear 128, and the proximal pinion gear 131 can engage with and drive the trocar gear 127. The trocar gear 127 may be operably coupled to the trocar 160 such that the trocar 160 is rotated by the trocar gear 127. The coax gear 128 may be operably coupled to an outer coax cannula 173 such that the coax cannula 173 is rotated by the coax gear 128. In the depicted embodiment, the trocar gear 127 and the coax gear 128 have a similar diameter and/or number of teeth such that the coax trocar 160 and the coax cannula 173 can be rotated at a similar speed. In other embodiments, the trocar gear 127 and the coax gear 128 may differ in diameter and/or number of teeth such that the trocar 160 and the coax cannula 173 can be rotated at different speeds. For example, the trocar gear 127 may have a smaller diameter and/or fewer teeth than the coax gear 128 resulting in the trocar 160 rotating at a higher speed than the coax cannula 173. An inverse configuration is also within the scope of this disclosure. In other embodiments, an intermediate gear may engage with the reduction gear 129 and the trocar gear 160 or the coax gear 128 to cause the trocar gear 160 and the coax gear 128 to rotate in opposite directions.

The gears 126, 127, 128, 129, 130, 131 may be formed from any suitable rigid or semi-rigid material, such as polycarbonate, acrylonitrile butadiene styrene, polycarbonate acrylonitrile butadiene styrene copolymer, nylon, acetal, polyethylene (e.g., high density polyethylene and/or low density polyethylene), silicone, thermoplastic elastomer, steel, stainless steel, aluminum, brass, ceramic, and combinations thereof. The polymers may be reinforced with other materials, such as glass or aramid fibers.

In some embodiments, a gear reduction ratio of the transmission 125 may range from about 100:1 to about 1:1, from about 80:1 to about 10:1, from about 50:1 to about 20:1, or from about 40:1 to about 30:1. In other words, the trocar 160 and the coax cannula 173 may be rotated at a range of from about 0 rpm to about 4,000 rpm, from about 0 rpm to about 1,000 rpm, from about 0 rpm to about 500 rpm, or from about 200 rpm to about 300 rpm. A delivered torque force may range from about 0.01 Nm to about 2 Nm, from about 0.5 Nm to about 1 Nm, or from about 0.5 Nm to about 0.75 Nm.

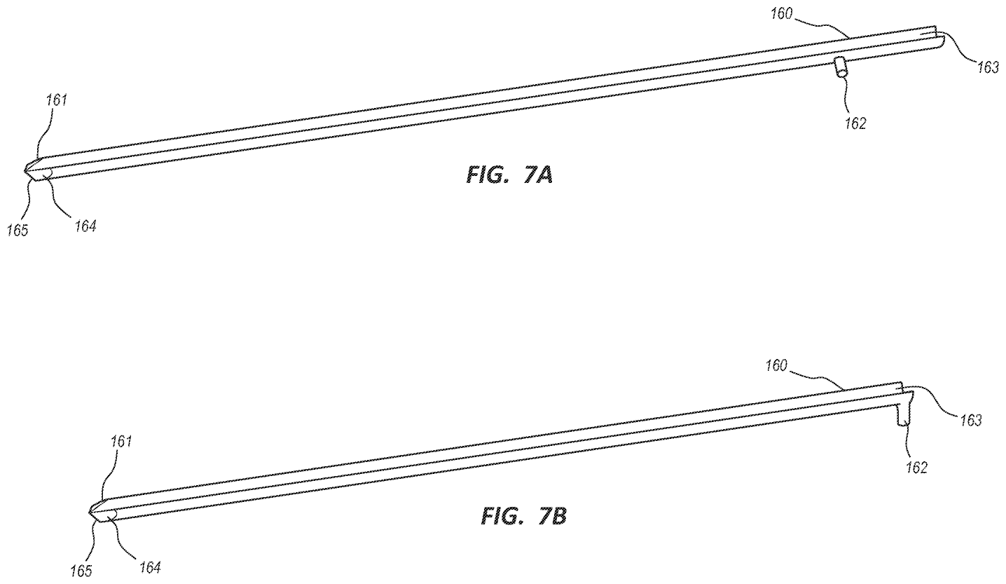

Referring to FIGS. 2 and 7A-7B, the trocar 160 may be an elongate rod having a penetrating tip 161. The penetrating tip 161 may include a plurality of facets 164 with cutting edges 165. The cutting edges 165 may be angled to allow for drilling of the trocar 160 into a bone. In some embodiments, the penetrating tip 161 may include spiral flutes. A laterally extending protrusion 162 may be disposed adjacent a proximal end of the trocar 160. In some embodiments the laterally extending protrusion 162 may be a pin as depicted in FIG. 7A. In other embodiments, a proximal end of the trocar 160 is bent at an approximately 90-degree angle relative to a longitudinal axis of the trocar 160 to form the lateral protrusion 162 as shown in FIG. 7B. The protrusion 162 may be configured to extend through a longitudinal slot 118 of a trocar tube 117. The trocar tube 117 extends proximally from the trocar gear 127 and is rotated as the trocar gear 127 is rotated resulting in rotation of the trocar 160. In an extended configuration the penetrating tip 161 extends distally beyond the outer coax cannula 173, as shown in FIG. 3A. And in a retracted configuration the penetrating tip 161 is disposed within the lumen 153 of the inner cannula 150, as shown in FIGS. 3B-1 and 3B-2. In certain embodiments, the trocar 160 may include a longitudinally extending groove or trough 163 as shown in FIGS. 7A and 7B. The groove 163 may have a substantially V-shape or U-shape and be configured for passage of a guidewire through the lumen 153 of the inner cannula 150 as described below.

An extension member 140 may be slidingly coupled to and extend proximally from the handle housing 111. The extension member 140 may also be slidingly coupled to the trocar tube 117. The extension member 140 is depicted to include an end cap 141 coupled to a proximal end of the extension member 140 and having a hollow distally extending portion. The end cap 141 may include a passage 145 through an end wall in axial alignment with the trocar 160 and configured for passage of a guidewire through the bone biopsy device 100 when in use, as will be described below. When the extension member 140 is displaced from a proximal position toward a distal position (e.g., by moving the extension member 140 distally), a distal end of the end cap 141 engages the protrusion 162 to displace the trocar 160 from the retracted configuration toward the extended configuration as the end cap 141 slides over the trocar tube 117. The extension member 140 may be selectively locked in the distal position by rotating the extension member 140 in a first direction such that an extension locking member 142 rotationally engages with a distally facing surface of an inner flange 149 of the handle housing 111. As depicted, the extension locking member 142 is disposed on an outer surface of the extension member 140. In other embodiments, the locking member 142 may form any other type of selective locking engagement, such as a snap fit, a press fit, a bayonet lock, etc.

The extension member 140 may be unlocked from the distal position when the extension member 140 is rotated in a second direction to align the locking member 142 with a gap between portions of the inner flange 149. When aligned, the locking member 142 can pass through the gap as the extension member 140 is displaced proximally. A resilient member or compression spring 146 may be disposed within the hollow portion of the end cap 141 with a distal end contacting a proximal end of the trocar tube 117 and a proximal end contacting the end wall of the end cap 141. The resilient member 146 can be compressed when the extension member 140 is displaced to the distal position. When the extension member 140 is unlocked from the distal position, the resilient member 146 may decompress and apply a proximally directed force to the extension member 140 to bias and/or cause the extension member 140 and the trocar 160 to be displaced proximally to the proximal position and retracted configuration, respectively. An inner flange or washer 144 of the extension member 140 may engage the protrusion 162 to displace the trocar 160 proximally. In certain embodiments, the trocar 160 may be slightly retracted as the extension member 140 is rotated and prior to displacement to the retracted configuration by the spring 146. This slight retraction may cause the trocar tip 161 to break free from tissue allowing the spring 146 to have a lower spring force.

In the illustrated embodiment, the handle housing 111 and the extension member 140 include indicia to indicate a status of the extension member, locked or unlocked. For example, the handle housing 111 may include an arrow 115 disposed at a proximal end of the handle housing 111 and the extension member 140 may include symbols 116 of a locked lock and an unlocked lock that are circumferentially spaced apart. When the extension member 140 is in a locked distal position the arrow 115 aligns with the locked lock symbol 116. When the extension member 140 is in an unlocked distal position following rotation of the extension member 140, the arrow 115 is aligned with the unlocked lock symbol 116. Other suitable indicia are contemplated within the scope of this disclosure.

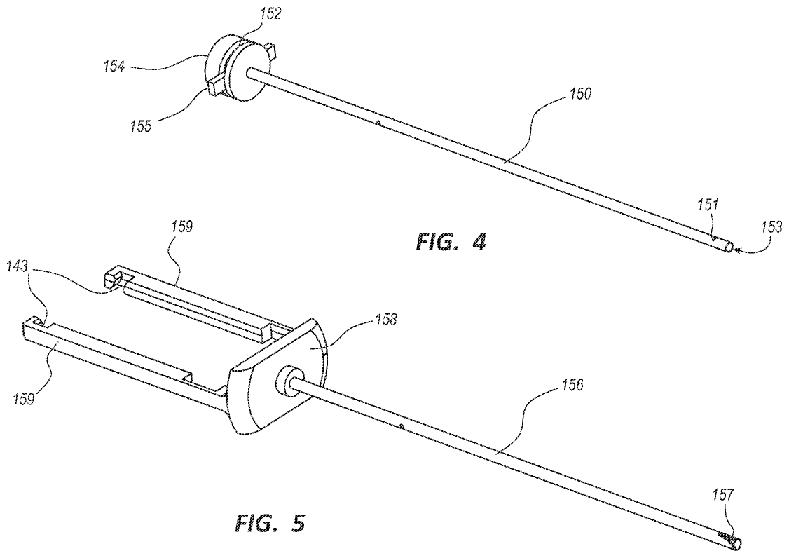

The inner cannula 150, as depicted in the illustrated embodiment of FIGS. 2 and 4, comprises a tube having a lumen 153. The trocar 160 can be coaxially disposed within the lumen 153. In some embodiments, a distal end may be sharpened to more easily penetrate tissue. The inner cannula 150 may be formed from any suitable material, such as stainless steel, titanium, titanium-nickel alloy, etc. As shown in the illustrated embodiment of FIGS. 2 and 4, the inner cannula 150 is fixedly coupled to a hub 154 and extends distally from the handle assembly 110. In some embodiments, the inner cannula 150 can be configured to receive a core tissue sample during a biopsy procedure. The inner cannula 150 includes a transverse slot 151 disposed adjacent a distal portion of the inner cannula 150 and communicating with the lumen 153. The hub 154 can be fixedly coupled to a proximal portion of the inner cannula 150. The hub 154 may include a circumferential channel 152 configured to receive an inner flange of the handle housing 111 to axially retain the inner cannula. One or more lugs 155 may radial outwardly extend from the hub 154 into a recess of the inner flange of the handle housing 111 to prevent the inner cannula 150 and the hub 154 from rotating.

In the depicted embodiment of FIGS. 2 and 5, at least a portion of the inner cannula 150 is coaxially disposed within the intermediate cannula 156. The intermediate cannula 156 is a tube including a part-off tab 157 disposed adjacent a distal end. The part-off tab 157 extends distally and is configured to pass through the transverse slot 151 and into the lumen 153 of the inner cannula 150. A hub 158 is fixedly coupled to a proximal end of the intermediate cannula 156. The hub 158 may include at least one proximally extending arm 159. In the depicted embodiment the hub 158 includes two proximally extending arms 159. A proximal portion of the arms 159 extends along the extension member 140 when the extension member 140 is in the distal position as shown in FIG. 3A. When the extension member 140 is in the proximal position, notches 143 in the arms 159 are configured to engage with the locking member 142 as the extension member 140 is rotated in the second direction, as shown in FIG. 3C. In doing so, the locking member 142 can apply a distally directed force to the arms 159 causing the hub 158 and the intermediate cannula 156 to be displaced distally. In other embodiments, proximal ends of the arms 159 may engage with the locking member to displace the hub 158 distally.

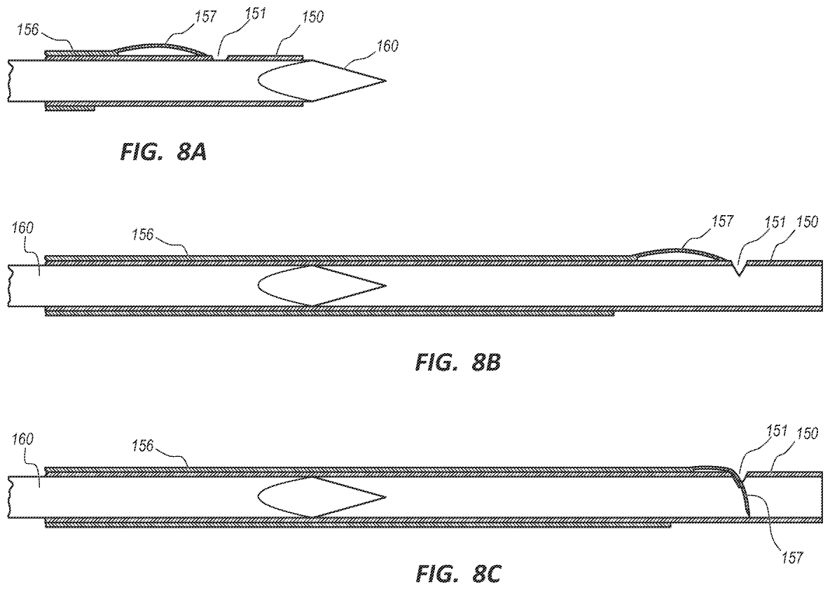

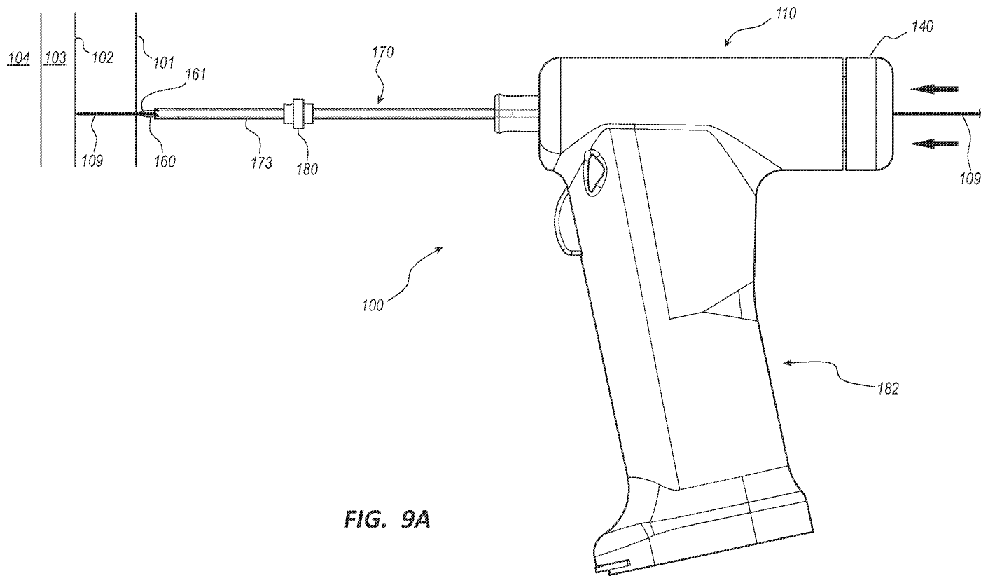

As shown in FIGS. 8A-8C, when the intermediate cannula 156 is displaced distally relative to the inner cannula 150, and the trocar 160 is retracted, the part-off tab 157 can pass through the transverse slot 151 and into the lumen 153 of the inner cannula 150. As the part-off tab 157 enters the lumen 153, it is configured to cut or sever a core tissue sample that is disposed within the lumen 153. Additionally, the part-off tab 157 can retain the tissue sample within the lumen 153. The part-off tab 157 can be retracted from the lumen 153 when the extension member 140 is rotated in a second direction. In certain embodiments, a resilient member may apply a proximally directed force to the hub 158 causing the intermediate cannula 156 and the hub 158 to be displaced proximally. In the illustrated embodiment, the locking member 142 may selectively couple with notches 143 of the arms 159 to proximally displace the intermediate cannula 156 when the extension member 140 is rotated in the first direction. For example, the locking member 142 may threadingly engage and disengage the arms 159. When the intermediate cannula 156 is displaced proximally, the part-off tab 157 is retracted from the lumen 153 allowing the core tissue sample to be expelled from the lumen 153 by the trocar 160 as will be described later.

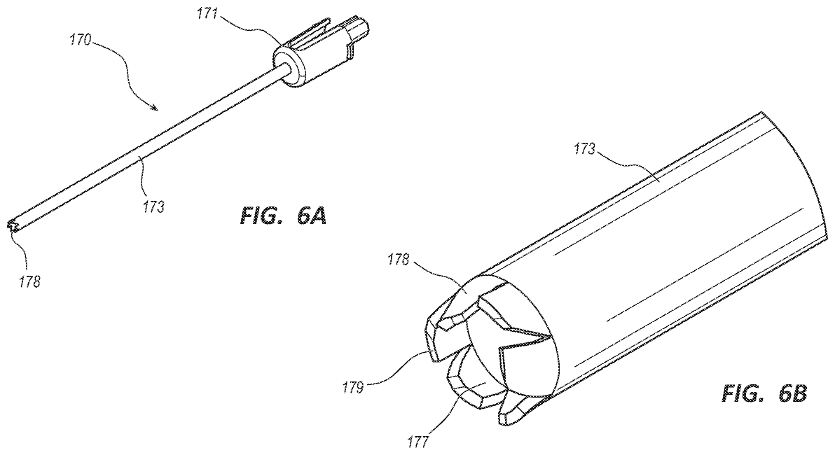

As depicted in the illustrated embodiment of FIGS. 2 and 6A-6B, the coax assembly 170 may be selectively coupled to the coax gear 128. The coax assembly 170 includes an outer coax cannula 173 fixedly coupled to a coax connector 171. The coax connector 171 may include a coupling member configured to mate with a retention member of the coax gear 128. In some embodiments, sides of the coax connector 171 are compressed (e.g., pushed inward) and pulled distally to disengage from the coupling member. For instance, the coupling member may include two proximal extending arms with at least one gap disposed between the arms. Radial inwardly extending hooks can be disposed at the proximal ends of the arms. The hooks can be configured to engage with the retention member. The retention member is shown as a radial inwardly extending ring. Other types of coupling mechanisms may be contemplated and are within the scope of this disclosure. For example, the coupling mechanism may be a bayonet fitting, a taper fitting, a threaded fitting, etc.

When coupled, the outer coax cannula 173 extends distally from and is rotated by the coax gear 128. The inner cannula 150 and the intermediate cannula 156 are coaxially disposed within a lumen 177 of the outer coax cannula 173. The inner cannula 150 may not extend beyond a distal end of the outer coax cannula 173. The outer coax cannula 173 may include a cutting tip 178, such as a trephine tip having a plurality of teeth 179 configured to rotate and saw a hole into a bone lesion and/or bone marrow when the outer coax cannula 173 is rotated. In some embodiments, the teeth 179 may be in alignment with a longitudinal axis of the outer coax cannula 173. In other embodiments, the teeth 179 may be alternatingly biased inwardly and outwardly relative to the longitudinal axis.

In the illustrated embodiment, a depth limiting member 180 is slidably coupled to the outer coax cannula 173. The depth limiting member 180 may be used to indicate an insertion depth of the outer coax cannula 173 into the patient that may correlate to a core tissue sample length. In some embodiments, the depth limiting member 180 may be rotated with the outer coax cannula 173. In other embodiments, the depth limiting member 180 may be held by a user while the outer coax cannula 173 is rotated to help guide the outer coax cannula 173 into the patient.

As depicted in the illustrated embodiment of FIG. 2, the power source 182 may be selectively disposed within the grip portion 113 of the handle housing 111. A removable cap 119 may retain the power source 182 within the handle housing 111. The power pack 182 may include a single battery or a plurality of batteries. The battery or batteries may be replaceable or rechargeable. In some embodiments, a controller may include a printed circuit board that is electrically coupled to the power source 182, the motor 122, and the motor activation switch 124. The controller can be configured to control activation and speed of the motor 122 when the motor activation switch 124 is actuated by the practitioner.

In certain embodiments, following a bone biopsy procedure, the power source 182 may be selectively removed from the bone biopsy device 100 and the handle assembly 110 and outer coax assembly 170 can be disposed of in a safe manner. As previously mentioned, the motor 122 can also be selectively removed from the handle assembly 110 if desired. When removed, the power source 182 and/or motor 122 may be refurbished for use in a subsequent procedure. Refurbishment may include cleaning, sterilizing, recharging or replacing the power source 182 and/or motor 122, etc. Alternatively, the power source 182 (and/or motor 122) may be disposed of in an environmentally friendly manner.

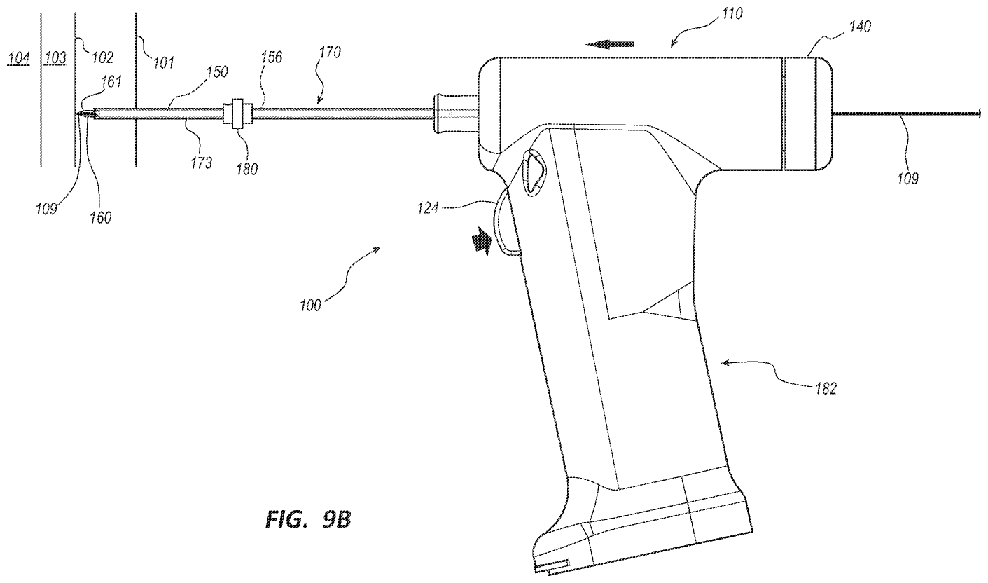

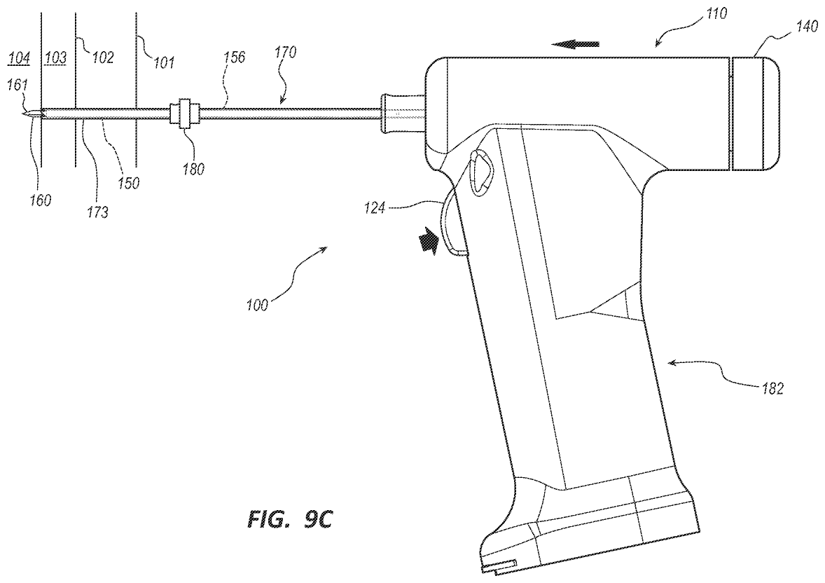

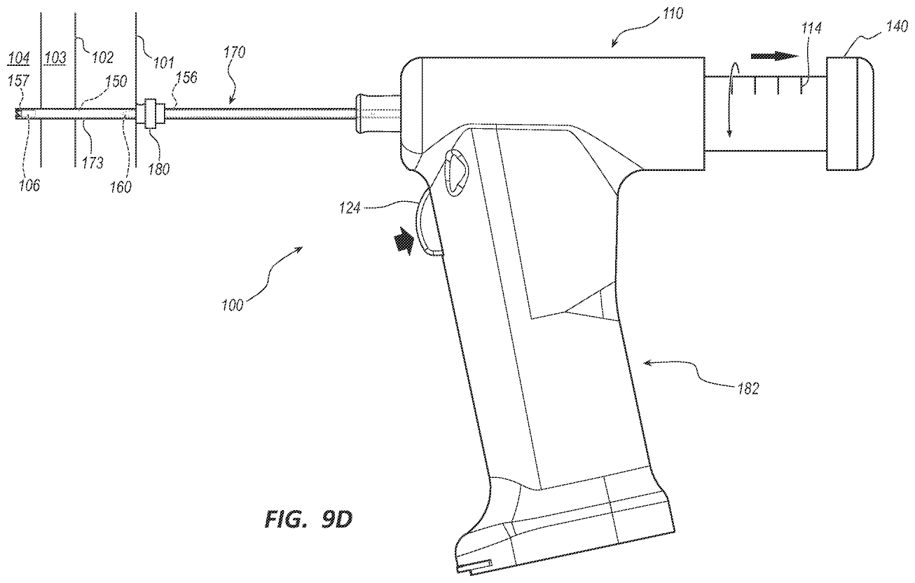

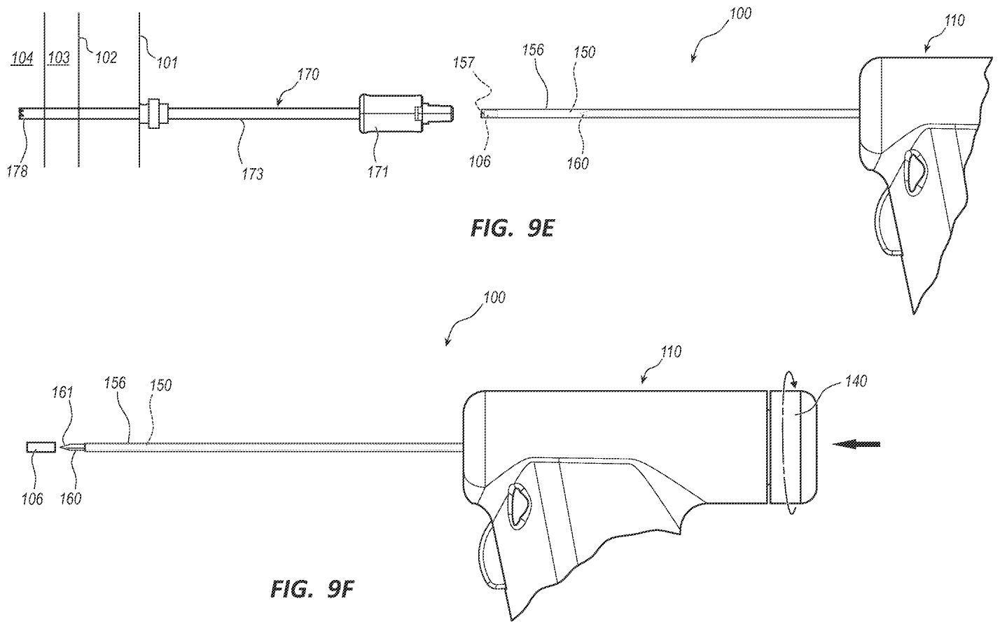

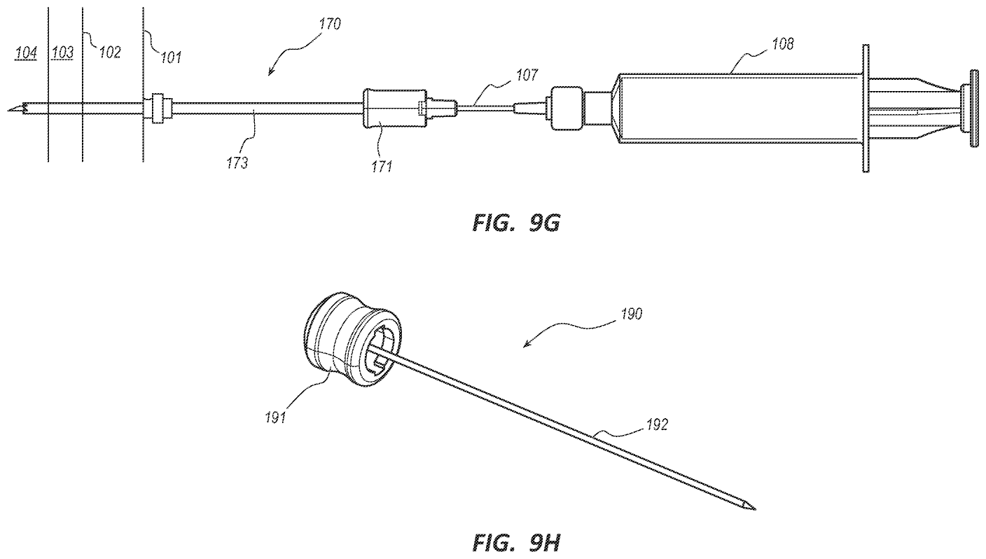

In use, the bone biopsy device 100 can be used to obtain a core tissue sample from a bone lesion and/or bone marrow. FIG. 9A illustrates the bone biopsy device 100 in a ready configuration. The power source 182 can be inserted into the handle assembly 110. The cap 119 can be coupled to the handle assembly 110 to retain the power source 182 within the handle assembly 110 and to prevent contamination of the power source 182 with body fluids. The coax assembly 170 may be coupled to the handle assembly 110. The extension member 140 can be displaced distally and locked in the distal position such that the trocar 160 is displaced from the retracted configuration to the extended configuration. In the extended configuration, the penetrating tip 161 extends distally beyond the outer coax cannula 173.

As depicted in FIG. 9B, the bone biopsy device 100 is inserted into the skin 101 toward the bone periosteum 102. The trocar 160 is in the extended configuration and the extension member 140 is locked in the distal position. The trocar 160, the inner cannula 150, the intermediate cannula 156, and the outer coax cannula 173 can be inserted through the patient's skin 101 as a unit until the penetrating tip 161 is adjacent the bone periosteum 102. The trocar 160 may be optionally inserted into the patient over a guidewire 109 that passes through the inner cannula via the trocar groove as previously described. The guidewire 109 may have been inserted using any suitable known technique prior to insertion of the bone biopsy device 100. The guidewire 109 can then be removed prior to rotating the outer coax cannula 173 when the penetrating tip 161 is adjacent the bone 102. In other instances, rotation of the outer coax cannula 173 and trocar 160 can begin prior to removal of the guidewire 109 to facilitate insertion of the trocar 160 through the skin 101.

FIG. 9C illustrates the penetrating tip 161 drilled through a bone cortex 103. To drill the penetrating tip 161 through the bone cortex 103, the motor 122 can be activated when the motor activation switch 124 is actuated by the practitioner. In some embodiments, the practitioner can control the motor speed through the motor activation switch 124. For example, the practitioner may partially actuate the motor activation switch 124 to run the motor 122 at a first speed and actuate the motor activation switch 124 further to run the motor 122 at a second speed, third speed, fourth speed, etc. The motor 122 can rotate the transmission to rotate the trocar 160 to drill through the bone cortex 103 until the cutting tip 178 of the outer coax cannula 173 is adjacent a bone lesion and/or bone marrow 104.

FIG. 9D illustrates the bone biopsy device 100 drilled into the bone lesion and/or bone marrow 104. The extension member 140 is unlocked and displaced to the proximal position. The extension member 140 is unlocked by rotation of the extension member in a first direction as previously described. The trocar 160 is displaced from the extended configuration to the retracted configuration. The motor 122 may be activated to rotate the outer coax cannula 173. The cutting tip 178 of the outer coax cannula 173 may saw a hole into the bone lesion and/or bone marrow 104. A core tissue sample 106 may be disposed within the inner cannula 150 as the cutting tip 178 saws the hole into the bone lesion and/or bone marrow 104. The part-off tab 157 may be actuated, as previously described (e.g., via rotation of the extension member 140), to cut or sever the core tissue sample 106 from the bone lesion and/or bone marrow 104. In some embodiments, a core tissue sample length may have been determined by the location of the depth limiting member 180 relative to the patient's skin 101.

In the illustrated embodiment, the handle assembly 110 also includes a core tissue sample length scale 114 disposed along the extension member 140. The scale 114 may include a plurality of indices, e.g., lines, spaced equidistance apart. In some embodiments, a distance between the lines may be 0.5 millimeter, one millimeter, two millimeters, etc. The scale 114, in cooperation with the extension member 140, may be used to determine the length of a core tissue sample that is contained within the lumen 153 of the inner cannula 150. For example, the extension member 140 and the trocar 160 may be displaced distally until the penetrating tip 161 engages with the core tissue sample 106 and the practitioner feels increased resistance to displace the extension member 140. A portion of the handle housing 111 may be adjacent to one line of the scale 114 that correlates with a length of the core tissue sample 106.

FIG. 9E depicts the coax assembly 170 decoupled from the handle assembly 110. The inner cannula 150, the intermediate cannula 156, and the trocar 160 are removed from the outer coax cannula 173 and the patient while the core tissue sample 106 is retained in the inner cannula 150 by the part-off tab 157. The outer coax cannula 173 may be left in the patient for obtaining subsequent core tissue samples and or biopsy samples. In other embodiments, the coax assembly 170 may not be decoupled from the handle assembly 110 and the outer coax cannula 173 may be removed from the patient with the handle assembly 110.

FIG. 9F illustrates the core tissue sample 106 ejected from the inner cannula 150 when the part-off tab 157 is unactuated as previously described and the extension member 140 is displaced from the proximal position to the distal position causing the trocar 160 to be displaced from the retracted configuration to the extended configuration. As the trocar 160 is displaced to the extended position, the penetrating tip 161 may push against the core tissue sample 106 to displace it distally from the inner cannula 150.

In some instances, as depicted in FIG. 9G, an aspiration needle 107 and an aspiration device (e.g., syringe, vacuum sample collection tube, or pump, etc.) 108 may be used to obtain a tissue sample of the bone lesion and/or bone marrow 104. For example, the needle may be inserted into the bone lesion and/or bone marrow 104 through the outer coax cannula 173 (which can be seated in the bone and/or patient after being decoupled from the handle assembly 110). The aspiration device 108 can then be used to aspirate a tissue sample of the bone lesion and/or bone marrow 104 through the needle.

In certain instances, a trocar assembly 190 may be selectively coupled to the coax assembly 170 to facilitate manual positioning of the coax assembly 170 prior to using the powered bone biopsy device. As illustrated in FIG. 9H, the trocar assembly 190 can include a handle member 191 and a trocar 192. In use, the trocar 192 may be inserted into the coax connector 171 and through the outer coax cannula 173 such that a distal tip of the trocar 192 extends beyond the outer coax cannula 173. The handle member 191 may also be coupled to the coax connector 171. The trocar assembly 190 and coax cannula 173 can then be moved and/or placed into a desired location (e.g., moved through the soft tissue). After proper placement is achieved, the trocar assembly 190 can be removed by uncoupling the handle member 191 from the coax connector 171 and removing the trocar 192 from the outer coax cannula 173. A powered bone biopsy device can thereafter be coupled with the outer coax cannula 173 and used to obtain a biopsy sample. For instance, an inner cannula 150, intermediate cannula 156, and trocar 160 coupled to a handle assembly 110 can be inserted into the outer coax cannula 173. The coax connector 171 can be coupled to the handle assembly 110 and a biopsy sample can thereafter be obtained as previously discussed.

In other embodiments, the trocar assembly 190 can be used to reposition or redirect the coax assembly 170 within the bone lesion and/or bone marrow to obtain subsequent tissue samples. For instance, after using the powered bone biopsy device (as previously discussed), the trocar assembly 190 can be inserted into and coupled to the coax assembly 170 to aid in manually repositioning and/or redirecting the coax assembly 170 prior to obtaining a subsequent core tissue sample or tissue sample using the powered bone biopsy device 100 or an aspiration needle 107.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in further embodiments, the feature can have a precisely perpendicular configuration.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A bone biopsy device, comprising:
a handle assembly, comprising:
a motor;
a transmission operably coupled to the motor;
an intermediate cannula;
an inner cannula coaxially disposed within the intermediate cannula; and
a penetration member operably coupled to the transmission and coaxially disposed within the inner cannula;
a coax assembly, comprising:
an outer coax cannula operably coupled to the transmission and coaxially disposed over the intermediate cannula; and
a power pack, comprising:
a power source.

2. The bone biopsy device of claim 1, wherein the handle assembly further comprises:
a handle housing; and
a motor activation switch.

3. The bone biopsy device of claim 1, wherein the transmission comprises a plurality of gears.

4. The bone biopsy device of claim 1, wherein the gear reduction ratio of the transmission ranges from 50:1 to 20:1.

5. The bone biopsy device of claim 1, wherein the penetration member further comprises a tip configured to drill into bone.

6. The bone biopsy device of claim 5, wherein the tip comprises a plurality of facets and angled cutting edges.

7. The bone biopsy device of claim 1, wherein the penetration member is a trocar.

8. The bone biopsy device of claim 1, wherein the penetration member further comprises a groove extending from a proximal end to a distal end.

9. The bone biopsy device of claim 8, wherein the groove is V-shaped.

10. The bone biopsy device of claim 1, wherein the outer coax cannula comprises a trephine tip comprising a plurality of distally extending teeth, wherein a first tooth of the plurality of teeth is biased radially inward and an adjacent tooth is biased radially outward.

11. The bone biopsy device of claim 1, wherein the coax assembly further comprises a depth limiting member slidingly disposed on the outer coax cannula.

12. The bone biopsy device of claim 1, wherein the power pack further comprises:
a power pack case;
a controller; and
a connector.

13. The bone biopsy device of claim 1, wherein at least one of the power pack or the motor are selectively removeable from the handle assembly.

14. The bone biopsy device of claim 1, wherein at least one of the power pack is reusable or motor is reusable.

15. A bone biopsy device, comprising:
a handle assembly, comprising:
a motor;
a transmission operably coupled to the motor;
an intermediate cannula;
an inner cannula coaxially disposed within the intermediate cannula; and
a penetration member operably coupled to the transmission and coaxially disposed within the inner cannula;
a coax assembly, comprising:
an outer coax cannula operably coupled to the transmission and coaxially disposed over the intermediate cannula; and
a power pack, comprising:
a power source,
wherein at least one of the power pack or the motor are selectively removeable from the handle assembly, and wherein at least one of the power pack is reusable or motor is reusable.

16. The bone biopsy device of claim 1, wherein the penetration member further comprises a groove extending from a proximal end to a distal end.

17. The bone biopsy device of claim 16, wherein the groove is V-shaped.

18. A method of obtaining a core tissue sample from a patient, comprising:
obtaining a bone biopsy device, comprising:
a handle assembly, wherein the handle assembly comprises:
an inner cannula;
an intermediate cannula;
a penetration member; and
a motor;
a coax assembly, wherein the coax assembly comprises an outer coax cannula; and
a power pack;
inserting the outer coax cannula, the inner cannula, the intermediate cannula, and the penetration member into the patient;
retracting the penetration member from an extended configuration to a retracted configuration;
further inserting the intermediate cannula and the inner cannula into at least one of a bone lesion or bone marrow;
obtaining the core tissue sample within the inner cannula;
removing the inner cannula, the intermediate cannula, and the penetration member from the patient; and
displacing the penetration member from the retracted configuration to the extended configuration to eject the core tissue sample from the inner cannula.

19. The method of claim 18, wherein inserting the outer coax cannula, the inner cannula, the intermediate cannula, and the penetration member into the patient comprises rotating the outer coax cannula, the inner cannula, the intermediate cannula, and the penetration member.

20. The method of claim 18, further comprising removing at least one of the power pack or motor from the biopsy device and re-using the at least one of the power pack or motor.
